(19) United States
(12) Patent Application Publication
(10) Pub. No.: US 2023/0191075 A1
(43) Pub. Date: Jun. 22, 2023

(54) SYSTEM AND METHOD FOR COMFORTING AN ANIMAL

(71) Applicant: Mathew Cleveland, Mesa, AZ (US)

(72) Inventor: Mathew Cleveland, Mesa, AZ (US)

(21) Appl. No.: 18/073,318

(22) Filed: Dec. 1, 2022

Related U.S. Application Data
(60) Provisional application No. 63/290,460, filed on Dec. 16, 2021.

(51) Int. Cl. A61M 21/02; A01K 1/00

(52) U.S. Cl. CPC A61M 21/02; A01K 1/0052; A01K 1/007; A61M 2021/0016

(57) ABSTRACT

An animal comfort accessory for use with an animal enclosure includes a cover configured to fit over the animal's enclosure to decrease noise and light for the animal in the enclosure. A scent blower moves air past a scent collector and delivers a soothing sent to the animal inside the enclosure. The scent blower and cover are contained in a pouch for convenient storage and travel. Additional accessories such as a speaker, camera, and video display may also be stored in the pouch and used with the animal to provide a comforting space for the animal.

### CROSS REFERENCE TO RELATED APPLICATION

This application claims the benefit of U.S. Provisional Patent Application No. 63/290,460 filed Dec. 16, 2021, which is incorporated by reference herein in its entirety.

### BACKGROUND OF THE INVENTION

### Field of the Invention

The invention related to pet containers and more particularly to a pet container that comforts the animal through dampening sound and providing a calming scent.

### Description of Related Art

It is known that there are many situations in which an animal will become irritated or anxious. Pets can become anxious when the pet is home alone, when there are loud noises such as cheering or fireworks, or if unfamiliar people are in the animal's area. In such circumstances, animal owners will frequently place their animals in an enclosed space such as a carrier or enclosure. The enclosed space serves as a kind of home base for the animal and can have a calming effect on anxious animals.

Numerous enclosures and systems exist to house pets while the owners are not present to care for the pets. The enclosures are designed to protect the pet in the absence of the owner and to prevent the pet from escaping and becoming lost. Pet hotels and pet sitting services provide care for pets while owners are away for extended periods of time. In addition, the pet accessory industry provides several types of pet beds. Many pets respond strongly to the scent of their owners. The owners' scent can calm the pet and give a feeling of normalcy to the pet in abnormal situations. U.S. Pat. No. 6,173,675 provides an aromatherapy mat for pets that allows a user to fill the mat with herbs or some other source of aroma, but there remains a need for a system and method of comforting animals by providing an enclosed space that is more conducive to animal comfort.

So as to reduce the complexity and length of the Detailed Specification, and to fully establish the state of the art in certain areas of technology, Applicant(s) herein expressly incorporate(s) by reference all of the following materials identified in each numbered paragraph below. The incorporated materials are not necessarily “prior art” and Applicant(s) expressly reserve(s) the right to swear behind any of the incorporated materials.

U.S. Pat. No. 6,173,675 to Licciardo, Rochine, Aromatherapy Mats for Pets, filed Nov. 12, 1998.

U.S. Pub. No. US 2005/0284418 to Benefiel, Tamara Lynn, Doggie Blanket Coat, filed Jun. 28, 2004.

U.S. Pub. No. US 2006/0266294 to Holmes, Cynthia and Crouch, Kay, Animal Sleeping Bag, filed May 26, 2005.

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), applicant(s) will amend the specification to expressly recite the essential material that is incorporated by reference as allowed by the applicable rules.

### BRIEF SUMMARY OF THE INVENTION

The present invention provides among other things an animal comfort accessory for use with an animal enclosure including a cover configured to fit over the animal's enclosure to decrease noise and light for the animal in the enclosure called the Kozy Canine Cover™. A scent blower moves air past a scent collector and delivers a soothing sent to the animal inside the enclosure. The scent blower and cover are contained in a pouch for convenient storage and travel. Additional accessories such as a speaker, camera, and video display may also be stored in the pouch and used with the animal to provide a comforting space for the animal.

It is an object of the invention to provide a comforting environment for an animal such as a dog or a cat.

It is another object of the invention to provide a sound dampening accessory for a conventional pet carrier or enclosure.

It is another object of the invention to comfort an animal by providing the scent of the animal's owner to the animal in the owner's absence.

It is another object of the invention to provide aromatherapy to an animal.

It is another object of the invention to provide a comforting space to an animal.

It is another object of the invention to provide an improved enclosure for enclosure training an animal.

It is another object of the invention to make animal owners more comfortable leaving their animals in their enclosure.

It is another object of the invention to provide a place where an animal can relax in a stressful situation.

It is another object of the invention to provide an accessory that will work with an existing enclosure or carrier that will improve the experience for the animal using the enclosure or carrier.

It is another object of the invention to deliver the scent of an animal's owner to the animal when the owner is not present.

It is another object of the invention to provide a home-like environment to an animal while traveling.

It is another object of the invention to provide a portable animal comfort apparatus.

The above and other objects may be achieved using devices involving an animal comfort accessory for use with an animal enclosure. A cover is provided that is configured to fit over the animal's enclosure, The cover may be made from a machine washable material and designed to fit a standard animal enclosure and may have a weighted fringe to keep the cover in place. The thickness of the cover may be such as to materially dampen the sound within the enclosure compared to the sound outside the enclosure. The cover has a scent portal that allows air to pass freely through the cover into the enclosure.

A scent blower having a fan may be detachably coupled to the cover to align the scent blower with the scent portal to blow a scent through the scent portal into the enclosure. In some embodiments, the scent blower and scent portal are located on the top of the enclosure. The scent blower includes a scent collector that has a desired scent, and the fan, powered by an AC power source, a battery, or a photovoltaic power source propels the scent of the scent collector through the scent portal into the enclosure. The scent blower may further comprise a conduit such as a flexible tube that contains the fan and the scent collector and directs the scented air to the scent portal. The conduit may be coupled to the cover to surround the scent portal and may use a sleeve about the conduit to do so. The scent blower may be connected to the cover by a plurality of snap connectors or a hook and loop fastener.

The scent blower may include a, air permeable chamber to maintain the scent collector in a desired position. The scent collector may be an item of clothing that has been worn by a person that is known to an animal that is to be enclosed in the enclosure and may be supplemented with an essential oil such as lavender.

The accessory may include a pouch that is coupled to the cover. The cover may be folded to be substantially contained within the pouch along with the scent blower. The pouch may also house a speaker, a camera, a video display, a food and/or water bowl, a wifi extender or other accessories. The pouch may be situated on the rear top side of the enclosure and may be coupled to the cover by at least one of stitches, a hook and loop connector, snaps, a zipper, grommets, and an adhesive.

The above and other objects may be achieved using methods involving comforting an animal in an enclosure by providing a cover that is configured to fit over the enclosure, the cover having a scent portal. A scent blower is detachably coupled to the cover about the scent portal. The scent blower has a fan and a scent collector. The scent collector may be placed into the scent blower by the user and engaging the fan propels the scent of the scent collector through the scent portal into the enclosure. The fan and the scent collector may be enclosed in a conduit. The scent collector may be supplement by applying an essential oil such as lavender to the scent blower.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning.

For purposes of this disclosure, the term “such as” has the special meaning of—including, but not limited to.

Absent such clear statements of intent to apply a “special” definition, it is the inventors' intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts a front perspective view of an accessory according to one or more embodiments of the invention draped about an enclosure

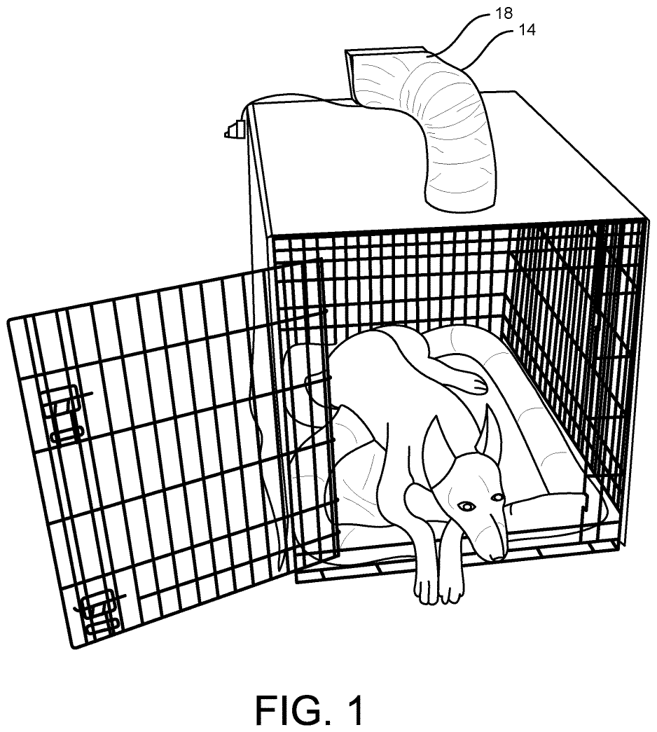

FIG. 2 depicts a front perspective view of an accessory with an upraised pocket according to one or more embodiments of the invention draped about an enclosure.

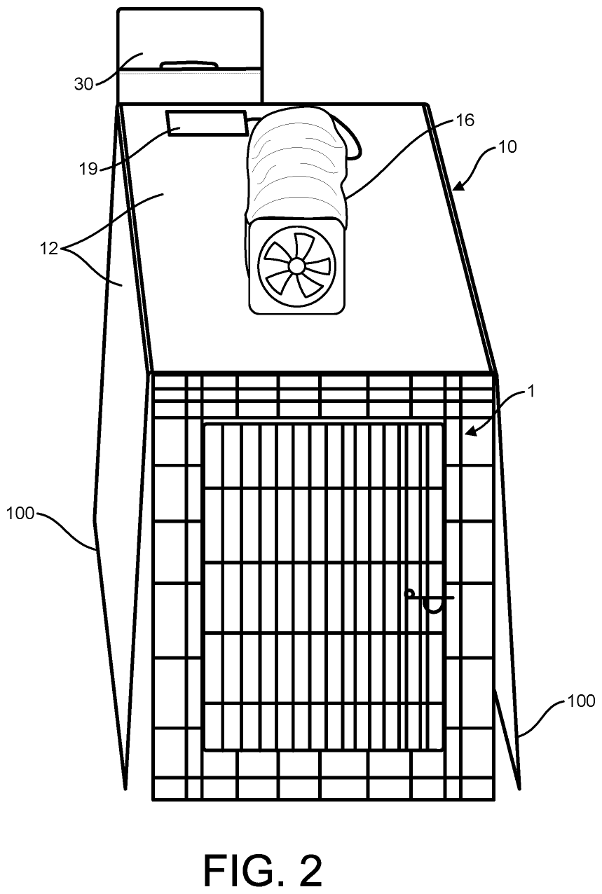

FIG. 3 depicts an overhead perspective view of the cover of an accessory according to one or more embodiments of the invention spread out flat.

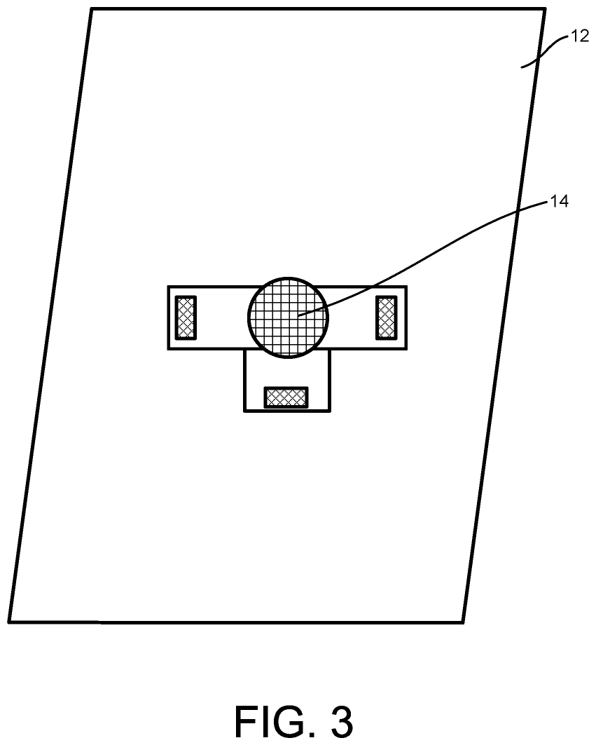

FIG. 4 depicts an overhead view of the scent portal on the cover of an accessory according to one or more embodiments of the invention.

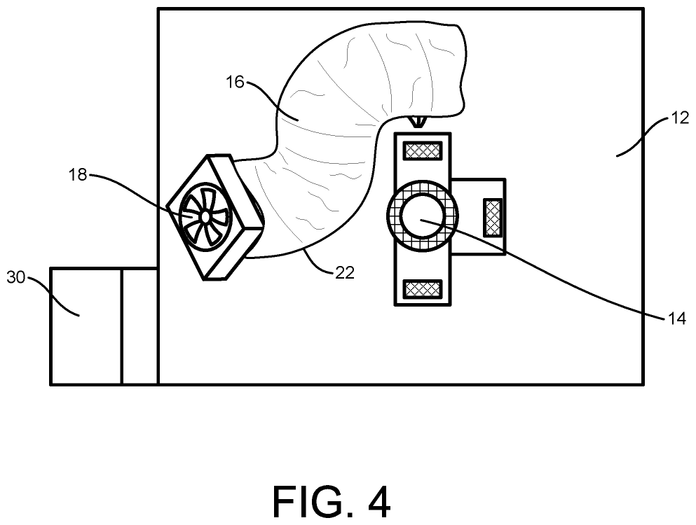

FIG. 5 depicts an overhead perspective view of a sleeve of an accessory according to one or more embodiments of the invention laid flat.

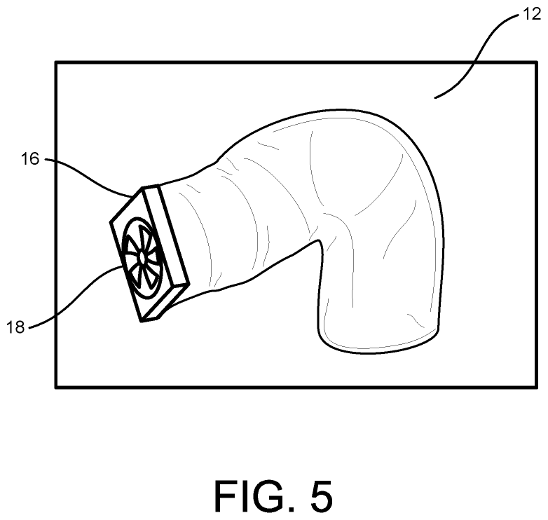

FIG. 6 depicts an overhead perspective view of the blower, conduit, and battery of an accessory according to one or more embodiments of the invention.

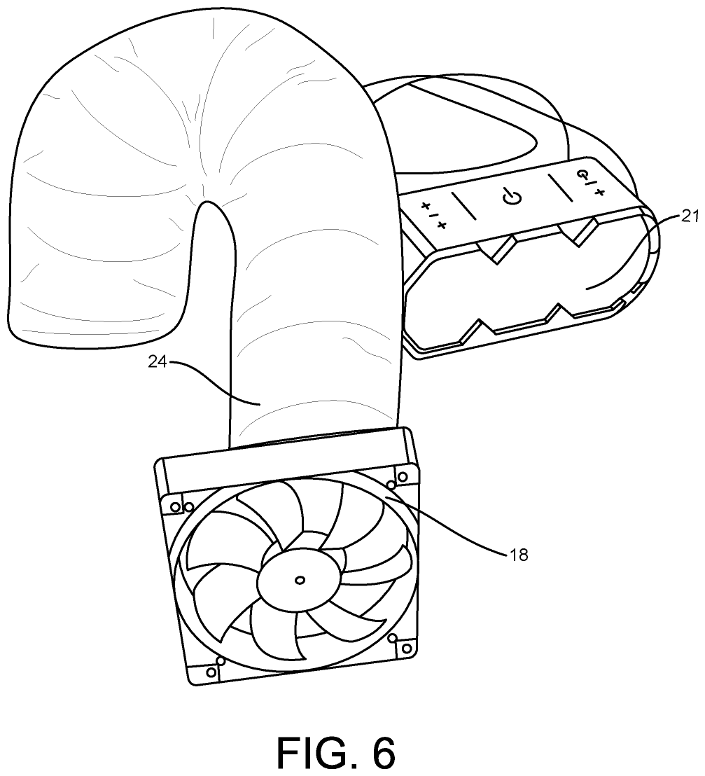

FIG. 7 depicts a side perspective view of the scent blower coupled to the cover of an accessory according to one or more embodiments of the invention.

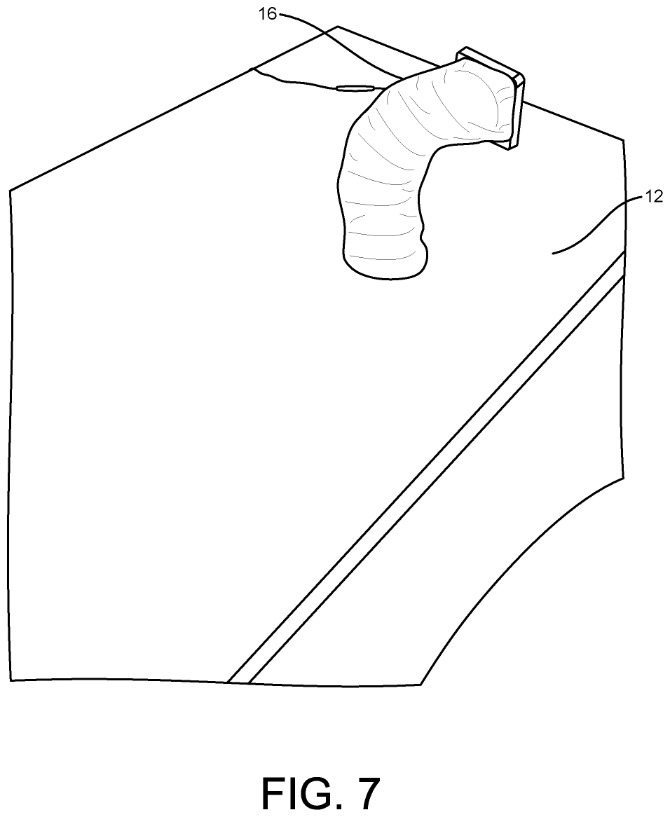

FIG. 8 depicts a rear perspective view of the scent blower coupled to the cover of an accessory according to one or more embodiments of the invention.

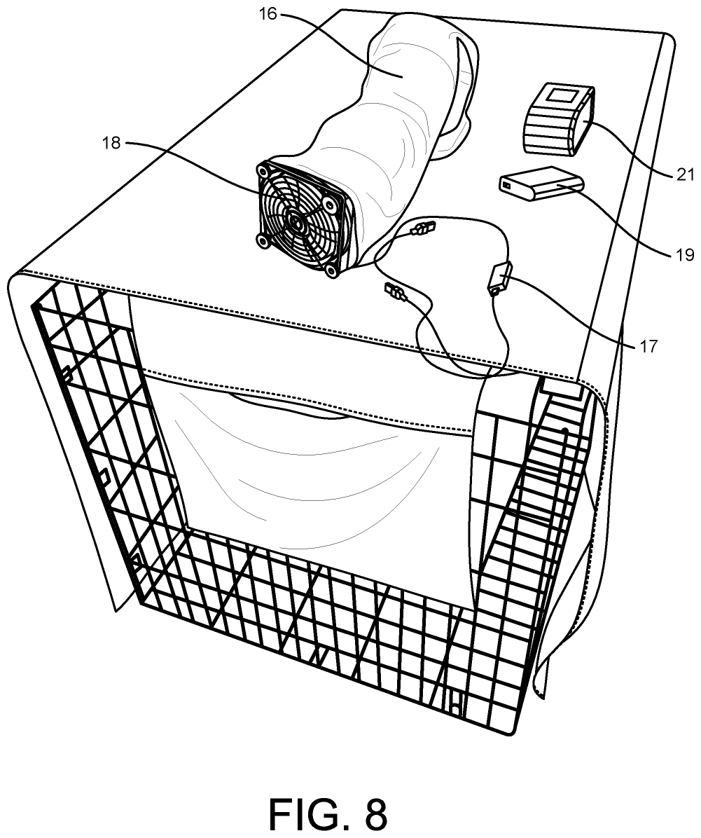

FIG. 9 depicts an overhead view of the cover and scent portal according to one or more embodiments of the invention.

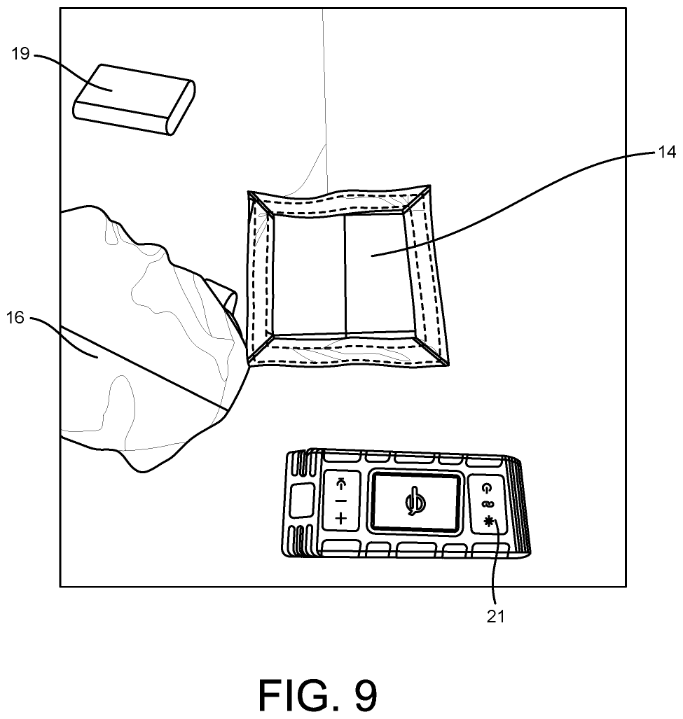

FIG. 10 depicts a closeup perspective view of the scent blower of an accessory according to one or more embodiments of the invention.

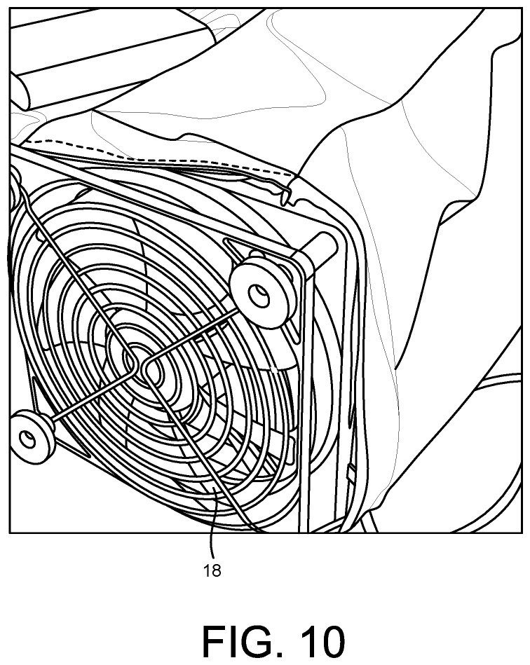

FIG. 11 depicts an overhead view of the battery and switch of an accessory according to one or more embodiments of the invention.

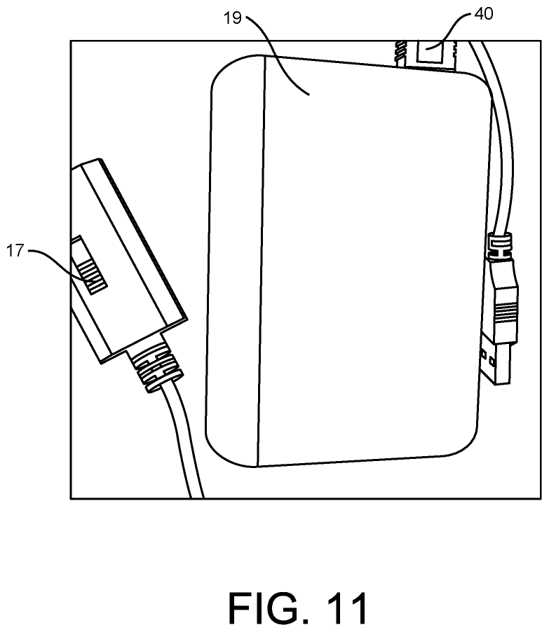

FIG. 12 depicts a front overhead perspective view of an accessory according to one or more embodiments of the invention.

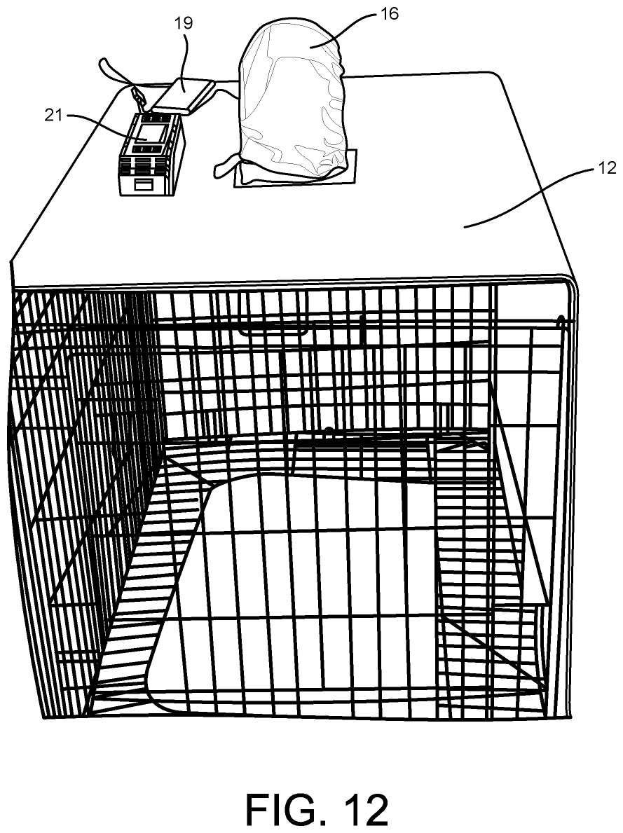

FIG. 13 depicts an overhead perspective view of an accessory according to one or more embodiments of the invention

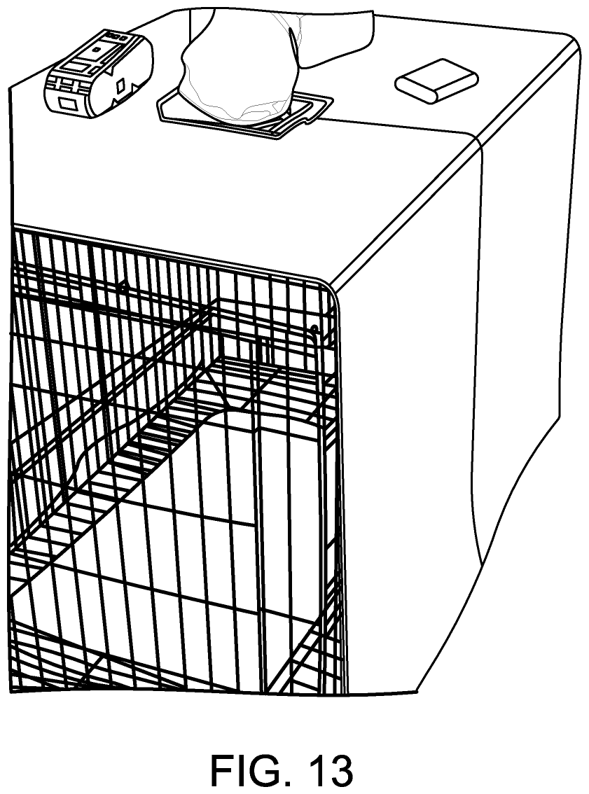

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention.

It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

In one application of the invention, an animal comfort accessory 10 is provided. The accessory 10 is configured to fit about an animal enclosure such as a kennel 1, crate, or pet carrier. The accessory may be sized to be used with a standard sized enclosure. Common standard sizes include enclosures for animals weighing between 40 and 60 lbs (30″×24″), 80 and 100 lbs (42″×30″), 100 and 150 lbs (60″×36″), and 150 and 180 lbs (72″×36″). Standard sizes are also available for smaller (<40 lbs) and larger (>200 lbs) and are known to those having skill in the art. The accessory 10 is effective for any animal that has an affinity for a particular noise level or scent.

The accessory includes a cover 12 that is configured to fit about the kennel 1. The cover 12 may be made of a machine washable material and have a thickness sufficient to material dampen the noise inside the enclosure compared to the noise outside the enclosure when the cover 12 is placed on the enclosure. To help the cover 12 remain in place, the cover may comprise a weighted fringe 100 or straps (not shown) that secure the cover 12 in place about the enclosure.

The cover 12 includes an air permeable, breathable scent portal 14 that allows scent laden air to pass through the cover 12 into the enclosure. The scent portal 14 may include a screen 15 that allows air to pass through the scent portal 14 but prevents larger items to fall through the scent portal 14. A scent blower 16 couples to the cover 12 about the scent portal 14. The scent blower 16 comprises a fan 18 that selectively moves air past a scent collector 20. The scent collector 20 may be any item conveying a scent, such as an article of clothing that has been worn by a person that is familiar to the animal to be placed into the enclosure. For example, a dog's owner may use a shirt, blanket, or other personal item conveying the owner's scent as the scent collector. The scent collector may be supplemented by an additional scent element such as an essential oil such as lavender. Lavender is a scent that is known to calm dogs and other animals.

The scent blower may also comprise a conduit 22. The conduit 22 is a space through which air is blown by the fan 18. The conduit 22 may be a volume such as a flexible tube 25. The conduit 22 houses the scent collector 20 and contains the air moved by the fan 18 past the scent collector 20 carrying some of the scent from the scent collector 20. An air permeable housing 26 to contain the scent collector 20 and hold it in place may be provided. The scent collector 20 may be placed between the fan 18 and the scent portal 14 or the fan 18 may be placed between the scent collector 20 and the scent portal 14 and draw air past the scent collector 20, through the fan 18, and then through the scent portal 14 into the enclosure. The air permeable housing may be coupled to the conduit 22 or the fan 18 depending upon the configuration of the system.

The scent blower 16 couples to the cover 12 about the scent portal 14. The scent blower 16 may be coupled to the cover 12 by any connecting mechanism known to those having skill in the art, such as hook and loop, snaps, buttons, clips, ties, or other connectors. In a particular embodiment the conduit and fan are contained within a sleeve 24. The sleeve 24 comprises four hook and loop connectors 32 situated about the conduit 22 like the four points of a compass. The cover 12 comprises loops 34 through which the hook and loop strap may be threaded and affixed by contacting the hook section of the connectors 32 to the loop section of the connectors 32. When connected, the fan 18 moves air past the scent collector 20 through the conduit 22 and the scent portal 14 into the enclosure. The fan 18 may be controlled by a switch 17 and powered by an AC power source 40, a battery 19, a photovoltaic panel or other power source. The battery 19 may also power a speaker 21 such as a Bluetooth speaker that can play soothing noises to the animal within the enclosure.

The cover 12 may also comprise a pouch 30. The pouch 30 may share a portion of fabric that is integral to the cover 12 or may be sewn or otherwise coupled to the cover 12. The cover 12 may be folded to fit within the pouch 30 along with the scent blower 16. The pouch may also be sized and configured to house a speaker 21 such as a Bluetooth speaker and at least one bowl (not shown) such as a water or food bowl for more convenient portability. The pouch 30 may include elastic straps or slots to hold items more securely within the pouch 30. The pouch 30 may be closed with a zipper or other closing mechanism known to those having skill in the art.

In practice a user will be able to use the accessory enclosed within the pouch 30. The user opens the pouch and removes the scent blower 16 and any other items that may be held within the pouch 30 such as a speaker 21, food and/or water bowl or other item. The user then unfolds or removes the cover 12 from the pouch 30 and drapes the cover 12 about the enclosure placing the scent portal 14 where desired. The scent blower 16 may already have a scent collector 20 or the user may place a scent collector 20 in the scent blower 16. The user may then couple the scent blower 16 to the cover 12 about the scent portal 14. The fan 18 of the scent blower 16 is engaged to move air past or through the scent collector 20 and into the enclosure through the scent portal 14.

Additional items may be included with the accessory such as a camera and a display that will display an image of the user to the animal in the enclosure. Animal stress and anxiety may be alleviated by providing a comforting environment for the animal. Animals are frequently comforted by quiet, dark spaces in the presence of their owner. Embodiments of the invention provide a comforting environment in a standard animal enclosure by, for example, deadening alarming sounds, limiting light, and providing the comforting scent of the animal's caretaker. Other soothing sights, sounds, and scents may also be added if desired.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, the disclosed subject matter is in no way limited to a particular methodology, protocol, and/or reagent, etc., described herein. As such, various modifications, or changes to, or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

I claim:

1. An animal comfort accessory for use with an animal enclosure, the accessory comprising:
a cover that is configured to fit over the enclosure; the cover having a scent portal; and
a scent blower coupled to the cover about the scent portal, wherein the scent blower comprises a fan and at least one scent collector such that the fan propels the scent of the scent collector through the scent portal into the enclosure.

2. The accessory of claim 1, wherein the scent blower further comprises a conduit.

3. The accessory of claim 2, wherein the conduit is at least one of:
coupled to the cover to surround the scent portal;
comprised of a flexible tube;
situated in a sleeve;

[symbol]

4. The accessory of claim 2, wherein the cover is coupled to the scent blower via at least one of snap connectors, hook and loop fasteners,

5. The accessory of claim 1 wherein the scent blower is at least one of:
comprised of an air permeable chamber to contain the scent collector;
comprised of an essential oil;
positioned to be on top of the enclosure;
detachable;

[symbol]

6. The accessory of claim 1, wherein the scent collector is an item of clothing that has been worn by a person that is known to an animal that is to be enclosed in the enclosure.

7. The accessory of claim 1, wherein the cover has at least one of the following characteristics:
has a thickness sufficient to materially dampen outside noises;
is machine washable;
further comprises a weighted fringe;
is sized to fit a standard enclosure size.

8. The accessory of claim 1, further comprising a pouch that is at least one of:
coupled to the cover;
foldable to be substantially contained within the pouch along with the scent blower;
configured to contain a speaker;
configured to contain at least one bowl.

9. The accessory of claim 8 wherein the enclosure has a top, a bottom, a front, and a back, and wherein the pouch is situated on the cover to be on the back top of the enclosure.

10. The accessory of claim 8 wherein the pouch is coupled to the cover by at least one of stitches, a hook and loop connector, snaps, a zipper, grommets, and an adhesive.

11. The accessory of claim 1 wherein the at least one scent collector is scented with at least one of the scent of a person that is known to an animal that is to be enclosed in the enclosure, lavender, vanilla, and an essential oil.

12. The accessory of claim 3 wherein the sleeve is detachably coupled to the cover.

13. The accessory of claim 1 wherein the fan is powered by an AC electrical connection, a battery, or photo voltaic power.

14. The accessory of claim 1 further comprising a camera detachably coupled to the cover and in wireless communication with a network for monitoring within the enclosure.

15. The accessory of claim 1 further comprising a wife extender.

16. A method for comforting an animal in an enclosure, the method comprising the acts of:
providing a cover that is configured to fit over the enclosure; the cover having a scent portal;
coupling a scent blower to the cover about the scent portal, wherein the scent blower comprises a fan and at least one scent collector having a scent; and
propelling the scent of the scent collector through the scent portal into the enclosure.

17. The method of claim 16 further comprising providing a conduit as a part of the scent blower to enclose the fan and the scent collector.

18. The method of claim 16, further comprising applying an essential oil within the scent blower.

19. The method of claim 16, wherein the scent blower is detachable.
